(19) United States
(12) Patent Application Publication
(10) Pub. No.: US 2009/0234128 A1
(43) Pub. Date: Sep. 17, 2009

(54) Process for the Preparation of Intermediates of Rosiglitazone, Rosiglitazone and New Polymorphic Forms Thereof

(76) Inventor: Ernesto Duran Lopez, Castellbisbal (ES)

Correspondence Address: FROMMER LAWRENCE & HAUG, 745 FIFTH AVENUE- 10TH FL., NEW YORK, NY, 10151, US

(21) Appl. No.: 12/282,013

(22) PCT Filed: Mar. 8, 2007
(86) PCT No.: PCT/IB2007/002824

§ 371 Date: Jan. 14, 2009

Related U.S. Application Data
(60) Provisional application No. 60/780,358, filed on Mar. 8, 2006.

(51) Int. Cl. C07D 417/10

(52) U.S. Cl. 546/269.7

(57) ABSTRACT

The invention relates to a polymorphic form of 5-(4-[2-(N-methyl-N-(2-pyridyl)amino)ethoxy]benzylidene)-2,4-thiazolidinedione (Formula (I)): to a process for its preparation and to the use of such compound for preparing rosiglitazone in the form of a free base or a salt thereof. The invention also relates to a polymorphic form of rosiglitazone in the form of a free base, to a process for its preparation and to the use of such polymorph for preparing a salt of rosiglitazone. The invention also relates to a process of preparing a polymorphic form of a rosiglitazone salt.

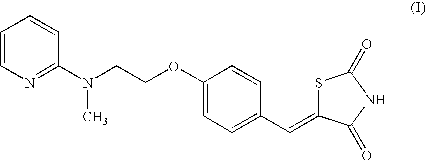

### REFERENCE TO RELATED APPLICATIONS/INCORPORATION BY REFERENCE

This application claims priority to U.S. Provisional Application 60/780,358, filed on 8 Mar. 2006.

Any foregoing applications, and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

Citation or identification of any document in this application is not an admission that such document is available as prior art to the present invention.

### FIELD OF INVENTION

The invention relates to a polymorphic form of 5-(4-[2-(N-methyl-N-(2-pyridyl)amino)ethoxy]benzylidene)-2,4-thiazolidinedione which has the formula,

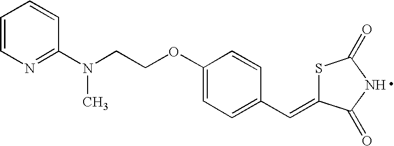

to a process for its preparation and to the use of such compound for preparing rosiglitazone in the form of a free base or a salt thereof. The invention also relates to a polymorphic form of rosiglitazone in the form of a free base, to a process for its preparation and to the use of such polymorph for preparing a salt of rosiglitazone.

### BACKGROUND OF THE INVENTION

Thiazolidinedione compounds such as troglitazone, pioglitazone and rosiglitazone have been used in clinical practice as oral hypoglycemic agents. Rosiglitazone maleate (5-(4-[2-(N-methyl-N-(2-pyridyl)amino)ethoxy]benzyl)-2,4-thiazolidinedione maleate):

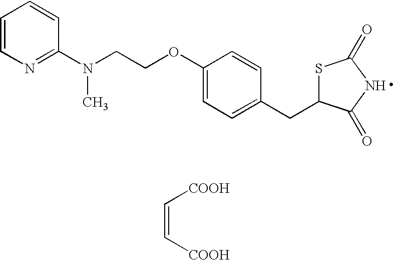

is an antidiabetic agent useful for the treatment of non-insulin dependent diabetes mellitus (NIDDM, also known as type II DM).

The rosiglitazone, or a tautomeric form and/or a pharmaceutically acceptable salt and/or a pharmaceutically acceptable solvate, are claimed in U.S. Pat. No. 5,002,953 (EP 306 228 B1), assigned to the Beecham Group.

U.S. Pat. No. 5,002,953 (EP 306 228 B1) also provides a process for the preparation of rosiglitazone base which comprises reacting 4-[2-(N-methyl-N-(2-pyridyl)amino)ethoxy]benzaldehyde and 2,4-thiazolidinedione to provide 5-(4-[2-(N-methyl-N-(2-pyridyl)amino)ethoxy]benzylidene)-2,4-thiazolidinedione which was then catalytically reduced.

Rosiglitazone maleate is specifically referred to in U.S. Pat. No. 5,741,803 (EP 658 161 B 1), assigned to the SmithKline Beecham Co. in addition to a process for preparing it, which comprises treating rosiglitazone with maleic acid.

The polymorphism of rosiglitazone maleate is referred to in SmithKline Beecham's PCT WO 00/64892, WO 00/64893 and WO 00/64896, in Dr. Reddy's PCT WO 02/26737 and in Chemi's U.S. Patent Application Publication 2005-0014798 A (EP 1 468 997 A) whereas SmithKline Beecham's PCT WO 99/31093, WO 99/31094, WO 99/31095 each refers to distinct hydrates of rosiglitazone maleate.

X-Ray crystal data for the enantiomer (R)-rosiglitazone are detailed in the paper published in J. Chem. Soc. Perkin Trans. (1), 1994, 3319-3324.

However, the X-ray powder diffraction pattern of 5-(4-[2-(N-methyl-N-(2-pyridyl)amino)ethoxy]benzylidene)-2,4-thiazolidinedione has not been previously described nor for racemic rosiglitazone base.

It is known in fact that many organic compounds may exist in the form of a plurality of different crystalline structures, which exhibit different physical properties. Chemical stability of the solid form and solubility in organic solvents can be dramatically affected by the crystalline structure, and changes in these properties have important effects in both yield and purity during the synthesis. Obtaining pure crystalline forms not only for the final pharmaceutical form, but also for its intermediates, is extremely useful because through these crystalline forms precise control of the process parameters is possible. Therefore, there is still a need in the art to develop processes for forming different crystalline forms of rosiglitazone and derivatives and intermediate compounds thereof.

### SUMMARY OF THE INVENTION

The present invention, inter alia, addresses a need in the art to develop processes for forming different crystalline forms of rosiglitazone, derivatives and intermediate compounds thereof by providing processes which yield consistently the same polymorphic form of 5-(4-[2-(N-methyl-N-(2-pyridyl)amino)ethoxy]benzylidene)-2,4-thiazolidinedione and 5-(4-[2-(N-methyl-N-(2-pyridyl)amino)ethoxy]benzyl)-2,4-thiazolidinedione and affords the synthesis of rosiglitazone maleate with high yield and pharmaceutically acceptable purity.

One aspect of the invention provides a novel polymorphic form of 5-(4-[2-(N-methyl-N-(2-pyridyl)amino)ethoxy]benzylidene)-2,4-thiazolidinedione and a process for its preparation which comprises of precipitation in an alcohol solvent.

The invention also provides for the synthesis of a polymorphic form of 5-(4-[2-(N-methyl-N-(2-pyridyl)amino)ethoxy]benzyl)-2,4-thiazolidinedione (rosiglitazone) from 5-(4-[2-(N-methyl-N-(2-pyridyl)amino)ethoxy]benzylidene)-2,4-thiazolidinedione via non-catalytic reduction.

The present invention also provides a polymorphic form of 5-(4-[2-(N-methyl-N-(2-pyridyl)amino)ethoxy]benzyl)-2,4-thiazolidinedione (rosiglitazone) in the form of a salt and a process for its preparation.

5-(4-[2-(N-methyl-N-(2-pyridyl)amino)ethoxy]benzyl)-2,4-thiazolidinedione as herein used is understood to mean rosiglitazone, in the form of a free base.

Terms “comprising” and “comprises” in this disclosure can mean “including” and “includes” or can have the meaning commonly given to the term “comprising” or “comprises” in US Patent Law. Terms “consisting essentially of” or “consists essentially of” if used in the claims have the meaning ascribed to them in US Patent Law. Other aspects of the invention are described in or are obvious from (and within the ambit of the invention) the following disclosure.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention. In the drawings:

FIG. 1: shows the powder X-ray diffraction spectrum of 5-(4-[2-(N-methyl-N-(2-pyridyl)amino)ethoxy]benzylidene)-2,4-thiazolidinedione.

FIG. 2: shows the IR spectrum of 5-(4-[2-(N-methyl-N-(2-pyridyl)amino)ethoxy]benzylidene)-2,4-thiazolidinedione.

FIG. 3: shows the powder X-ray diffraction spectrum of 5-(4-[2-(N-methyl-N-(2-pyridyl)amino)ethoxy]benzyl)-2,4-thiazolidinedione.

FIG. 4: shows the IR spectrum of 5-(4-[2-(N-methyl-N-(2-pyridyl)amino)ethoxy]benzyl)-2,4-thiazolidinedione

FIG. 5: shows the IR spectrum of rosiglitazone maleate.

FIG. 6: shows the powder X-ray diffraction spectrum of 4-[2-(N-methyl-N-(2-pyridyl)amino)ethoxy]benzaldehyde as a commercial product.

FIG. 7: shows the powder X-ray diffraction spectrum of rosiglitazone maleate.

### DESCRIPTION OF THE INVENTION

The present invention provides a novel polymorphic form of 5-(4-[2-(N-methyl-N-(2-pyridyl)amino)ethoxy]benzylidene)-2,4-thiazolidinedione and a process for its preparation which comprises of precipitation in an alcohol solvent.

In one embodiment of the invention, the process for preparing the polymorphic form of 5-(4-[2-(N-methyl-N-(2-pyridyl)amino)ethoxy]benzylidene)-2,4-thiazolidinedione comprises:
(a) reacting 4-[2-(N-methyl-N-(2-pyridyl)amino)ethoxy]benzaldehyde with thiazolidine-2,4-dione in a first organic solvent to form crude 5-(4-[2-(N-methyl-N-(2-pyridyl)amino)ethoxy]benzylidene)-2,4-thiazolidinedione;
(b) mixing the crude 5-(4-[2-(N-methyl-N-(2-pyridyl)amino)ethoxy]benzylidene)-2,4-thiazolidinedione with a second organic solvent to form a solution;
(c) mixing a third organic solvent with the solution of step (b) to precipitate a polymorphic form of 5-(4-[2-(N-methyl-N-(2-pyridyl)amino)ethoxy]benzylidene)-2,4-thiazolidinedione; and
(d) optionally, recrystallizing the polymorphic form of 5-(4-[2-(N-methyl-N-(2-pyridyl)amino)ethoxy]benzylidene)-2,4-thiazolidinedione in step (c).

In another embodiment of this invention, the first organic solvent is an aromatic solvent, the second organic solvent is an amido solvent; and the third organic solvent is an alcohol.

In yet another embodiment of this invention, the first organic solvent is toluene, the second organic solvent is N,N-dimethylformamide (DMF); and the third organic solvent is isopropanol.

An alternative embodiment of the process for preparing the polymorphic form of 5-(4-[2-(N-methyl-N-(2-pyridyl)amino)ethoxy]benzylidene)-2,4-thiazolidinedione

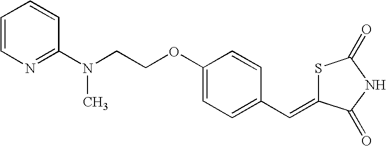

comprises:
(a) mixing the compound 5-(4-[2-(N-methyl-N-(2-pyridyl)amino)ethoxy]benzylidene)-2,4-thiazolidinedione with a first organic solvent to form a solution;
(b) mixing a second organic solvent with the solution of step (a) to precipitate a polymorphic form of 5-(4-[2-(N-methyl-N-(2-pyridyl)amino)ethoxy]benzylidene)-2,4-thiazolidinedione
   wherein the first organic solvent is an amido solvent and the second organic solvent is an alcohol.

In another form of the alternative embodiment process of the process for preparing the polymorphic form of 5-(4-[2-(N-methyl-N-(2-pyridyl)amino)ethoxy]benzylidene)-2,4-thiazolidinedione, the first organic solvent is N,N-dimethylformamide (DMF); and the second organic solvent is isopropanol.

In yet another form of the alternative embodiment process of the process for preparing the polymorphic form of 5-(4-[2-(N-methyl-N-(2-pyridyl)amino)ethoxy]benzylidene)-2,4-thiazolidinedione, the ratio of N,N-dimethylformamide (DMF)/isopropanol is about 1:2 to about 1:5.

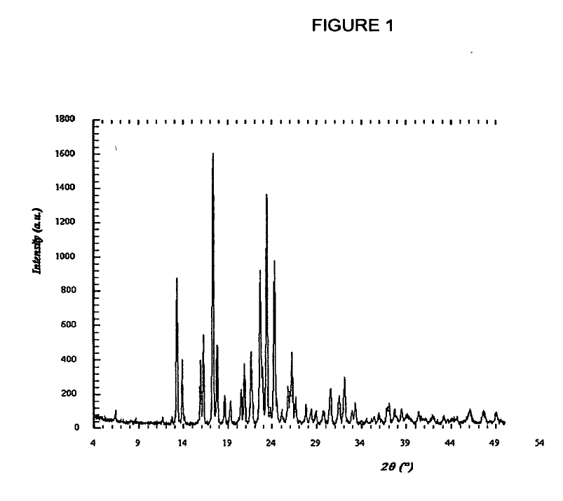

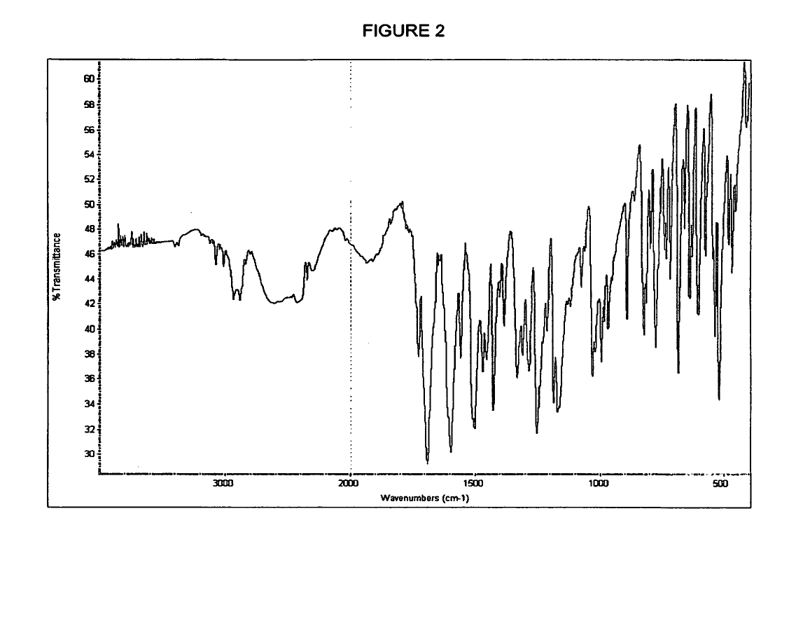

In still another form of the alternative embodiment process of the process for preparing the polymorphic form of 5-(4-[2-(N-methyl-N-(2-pyridyl)amino)ethoxy]benzylidene)-2,4-thiazolidinedione, the polymorphic form of 5-(4-[2-(N-methyl-N-(2-pyridyl)amino)ethoxy]benzylidene)-2,4-thiazolidinedione has:
(1) the X-ray powder diffraction (XRPD) characterized by the principal angles and relative intensities reported in FIG. 1; and
(2) the infrared absorption spectrum characterized by the principal absorptions reported in FIG. 2.

The invention also provides for the synthesis of a polymorphic form of 5-(4-[2-(N-methyl-N-(2-pyridyl)amino)ethoxy]benzyl)-2,4-thiazolidinedione (rosiglitazone) from 5-(4-[2-(N-methyl-N-(2-pyridyl)amino)ethoxy]benzylidene)-2,4-thiazolidinedione via non-catalytic reduction.

In one embodiment of the invention, the process of preparing the polymorphic form of rosiglitazone from 5-(4-[2-(N-methyl-N-(2-pyridyl)amino)ethoxy]benzylidene)-2,4-thiazolidinedione comprises:
(a) reacting 5-(4-[2-(N-methyl-N-(2-pyridyl)amino)ethoxy]benzylidene)-2,4-thiazolidinedione with a dihydropyridine carboxylate compound in a first organic solvent to form crude rosiglitazone;
(b) adding a second organic solvent to the crude rosiglitazone to form a solution and concentrating the solution;
(c) adding a third organic solvent to the concentrated solution of step (b) to form a suspension;
(d) isolating the polymorphic form of rosiglitazone from the suspension of step (c).

In another embodiment of this invention, the first organic solvent is an aromatic solvent, the second organic solvent is a heterocyclic solvent; and the third organic solvent is an alcohol.

In yet another embodiment of this invention, the first organic solvent is toluene, the second organic solvent is tetrahydrofuran (THF); and the third organic solvent is isopropanol.

An alternative embodiment for the process of preparing the polymorphic form of rosiglitazone comprises:
(a) mixing rosiglitazone with a first organic solvent to form a solution;
(b) mixing a second organic solvent with the solution of step (a) to form a suspension;
(c) isolating the polymorphic form of rosiglitazone from the suspension of step (b),
   wherein the first organic solvent is a heterocyclic solvent and the second organic solvent is an alcohol.

In another form of the alternative embodiment for the process of preparing the polymorphic form of rosiglitazone, the first organic solvent is an ether cyclic solvent and the second organic solvent is a C₁-C₄ alcohol.

In yet another form of the alternative embodiment for the process of preparing the polymorphic form of rosiglitazone, the first organic solvent is tetrahydrofuran (THF); and the second organic solvent is isopropanol.

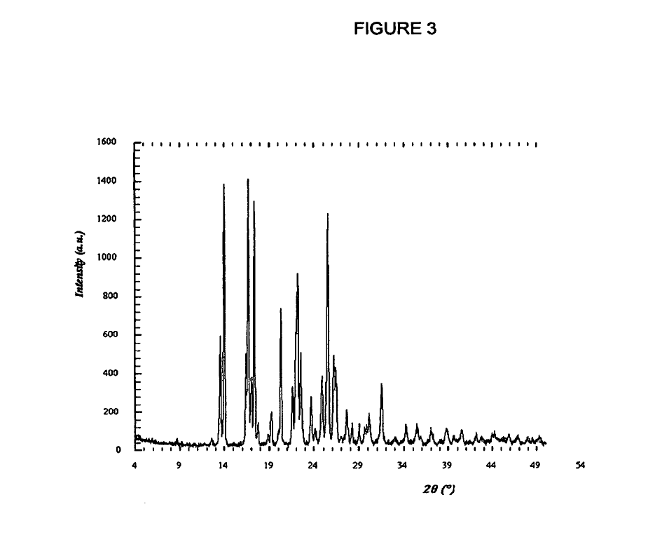

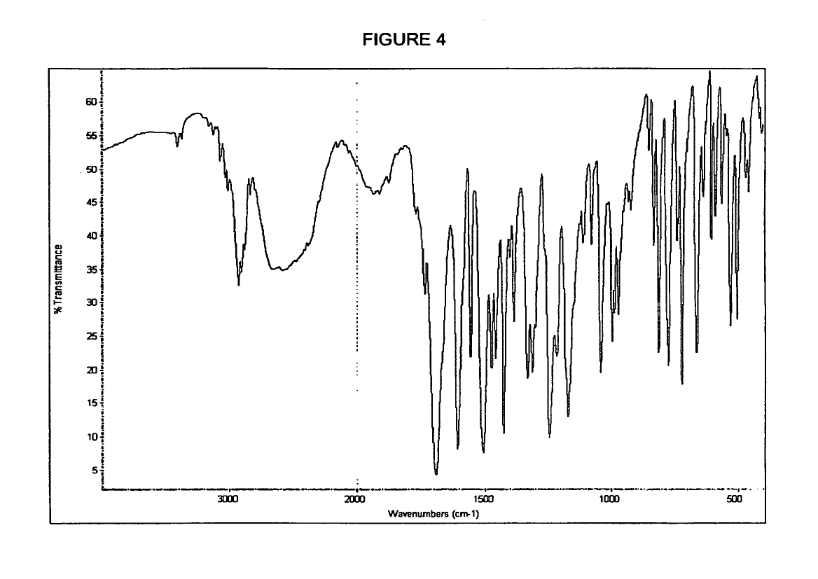

In still another form of the alternative embodiment for the process of preparing the polymorphic form of rosiglitazone, the polymorphic form of rosiglitazone has:
(1) the X-ray powder diffraction (XRPD) characterized by the principal angles and relative intensities reported in FIG. 3.
(2) the IR-spectrum characterized by the principal absorptions reported in FIG. 4.

The present invention also provides a polymorphic form of 5-(4-[2-(N-methyl-N-(2-pyridyl)amino)ethoxy]benzyl)-2,4-thiazolidinedione (rosiglitazone) in the form of a salt and a process for its preparation.

In one embodiment of the invention, the process of preparing the polymorphic form of a rosiglitazone salt comprises:
(a) reacting rosiglitazone with an organic acid in a first alcohol solvent to form a crude rosiglitazone salt;
(b) adding an organic acid of the salt and a second alcohol solvent to the crude rosiglitazone salt to form a suspension;
(c) filtering the suspension to obtain the polymorphic form of a rosiglitazone salt.

In one embodiment of this invention, the organic acid is a mono- or dicarboxylic acid, the first alcohol solvent is a C₁-C₆ alcohol and the second alcohol solvent is a C₁-C₆ alcohol wherein the first and second alcohol solvents are different.

In yet another embodiment of this invention, the organic acid is maleic acid, the first alcohol solvent is isopropanol and the second alcohol solvent is ethanol.

An alternative embodiment for the process of preparing the polymorphic form of a rosiglitazone salt comprises recrystallizing rosiglitazone salt with an organic acid of the salt in an alcohol solvent to form a polymorphic form of rosiglitazone salt, wherein the ratio of organic acid of the salt/rosiglitazone salt is about 1:2 to 1:20 by mole.

In another form of the alternative embodiment for the process of preparing the polymorphic form of a rosiglitazone salt, the organic acid of the salt is a mono- or di-carboxylic acid, the alcohol solvent is a C₁-C₆ alcohol and the ratio of organic acid of the salt/rosiglitazone salt is about 1:5 to 1:15 by mole.

In yet another form of the alternative embodiment for the process of preparing the polymorphic form of a rosiglitazone salt, the rosiglitazone salt is rosiglitazone maleate, the organic acid of the salt is maleic acid, the alcohol is ethanol and the ratio of amount of organic acid of the salt/rosiglitazone is about 1:6 by mole.

In still another form of the alternative embodiment for the process of preparing the polymorphic form of a rosiglitazone salt, the particle size distribution of the polymorphic form of rosiglitazone salt is:
(1) about 5 to about 15% of the total volume of polymorphic form of rosiglitazone salt having a particle size of between about 1.0 micrometer to about 1.5 micrometers;
(2) about 45 to about 55% of the total volume of polymorphic form of rosiglitazone salt having a particle size of between about 7.5 micrometers to about 8.5 micrometers; and
(3) about 85 to about 95% of the total volume of polymorphic form of rosiglitazone salt having a particle size of between about 37.0 micrometers to about 55.0 micrometers.

The polymorphic form of 5-(4-[2-(N-methyl-N-(2-pyridyl)amino)ethoxy]benzylidene)-2,4-thiazolidinedione, rosiglitazone and rosiglitazone maleate salt obtained are characterized by X-ray powder diffraction and IR.

Accordingly, the present invention discloses a polymorphic form of 5-(4-[2-(N-methyl-N-(2-pyridyl)amino)ethoxy]benzylidene)-2,4-thiazolidinedione characterized in that it shows:
(1) an X-ray powder diffraction (XRPD) characterized by the principal angles and relative intensities reported in Table 1 corresponding to FIG. 1.
(2) an infrared absorption spectrum characterized by the principal absorptions reported in FIG. 2.

In a further aspect the present invention also discloses a polymorphic form of rosiglitazone base characterized in that it shows:
(1) an X-ray powder diffraction (XRPD) characterized by the principal angles and relative intensities reported in Table 2 corresponding to FIG. 3.
(2) an IR-spectrum characterized by the principal absorptions reported in FIG. 4.

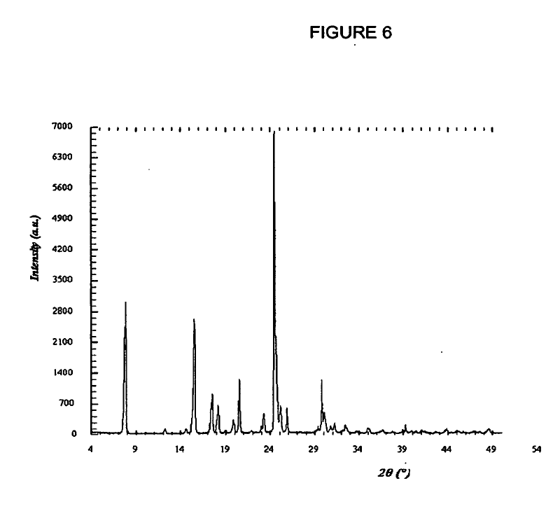

An X-ray powder diffraction (XRPD) pattern of commercial compound 4-[2-(N-methyl-N-(2-pyridyl)amino)ethoxy]benzaldehyde is also reported in this invention. The principal characteristic XRPD angles (2θ°) and relative intensities (in %) are reported in the table below corresponding to FIG. 6.

TABLE

| peak_position | peak_intensity |
|---|---|
| 7.500 | 299.0 |
| 7.640 | 1157.0 |
| 7.740 | 2452.0 |
| 7.838 | 2996.1 |
| 15.310 | 1031.1 |
| 15.469 | 2364.6 |
| 17.389 | 800.1 |
| 17.504 | 945.1 |
| 17.970 | 329.1 |
| 18.148 | 681.1 |
| 19.817 | 352.8 |
| 19.960 | 228.0 |
| 20.506 | 1265.0 |
| 23.228 | 470.2 |
| 24.445 | 6723.2 |
| 24.640 | 1772.0 |
| 25.000 | 500.0 |
| 25.111 | 671.2 |
| 25.838 | 602.8 |
| 29.776 | 1251.3 |
| 30.120 | 453.0 |
| 30.250 | 260.9 |
| 32.421 | 235.8 |
| 39.236 | 236.3 |

The invention is further described by the following non-limiting examples which further illustrate the invention, and are not intended, nor should they be interpreted to, limit the scope of the invention.

### EXAMPLES

### General Experimental Conditions

i. Particle Size Method

The particle size for rosiglitazone maleate was measured using a Malvern Mastersizer S particle size analyzer with an MS1 Small Volume Sample Dispersion Unit stirred cell. A 300RF mm lens and a beam length of 2.4 mm were used. Samples for analysis were prepared by dispersing a weighed amount of rosiglitazone maleate (approximately 50 mg) in 20 mL of Lecithin solution, previously prepared by mixture of 1.5 g of Soybean Lecithin and 200 mL of Isopar G. After sonication for 2 minutes, the suspension was delivered drop-wise to a background corrected measuring cell previously filled with Lecithin solution until the obscuration reached the desired level. Volume distributions were obtained for three times. After completing the measurements, the sample cell was emptied and cleaned, refilled with suspending medium, and the sampling procedure repeated again. For characterization, the values of D₁₀, D₅₀ and D₉₀ (by volume) were specifically listed, each one being the mean of the six values available for each characterization parameter.

### Example 1

### Preparation of 5-(4-[2-(N-methyl-N-(2-pyridyl)amino)ethoxy]benzylidene)-2,4-thiazolidinedione

100.0 g (390.2 mmol) of 4-[2-(N-methyl-N-(2-pyridyl)amino)ethoxy]benzaldehyde and 48.0 g (409.8 mmol) of thiazolidine-2,4-dione were suspended in 600 mL of toluene. 1.6 mL (19.7 mmol) of pyrrolidine and 1.1 mL (20.0 mmol) of glacial acetic acid were added over the stirred suspension. The mixture was heated to reflux temperature for 3 hours, with azeotropical distillation of water. The resulting suspension was cooled to 5° C. and filtered using a Büchner funnel. The filter cake was washed with 300 mL of toluene and 300 mL of isopropanol. 135.9 g of solid were obtained (98% yield, 97.11% purity by HPLC).

26.6 g of the crude solid were suspended in 80 mL of N,N-dimethylformamide. The suspension was heated to 120° C. to obtain a clear solution. The solution was cooled down to 80° C. and 266 mL of isopropanol were added. Precipitation of a yellowish solid was observed. The resulting suspension was cooled to 0° C. and filtered. The filter cake was washed with 80 mL of isopropanol. 25.4 g of recrystallized solid were obtained (95% yield, 98.75% purity by HPLC), m.p. 187-188.4° C.

The following characterization data was generated for the 5-(4-[2-(N-methyl-N-(2-pyridil)amino)ethoxy]benzylidene)-2,4-thiazolidinedione.

### A. X-Ray Powder Diffraction (XRPD)

The XRPD pattern is shown below in FIG. 1 and a summary of the characteristic XRPD angles (2θ°) and relative intensities (in %) are given in Table 1

TABLE 1

| peak_position | peak_intensity |
|---|---|
| 6.408 | 109.3 |
| 11.633 | 61.4 |
| 12.672 | 64.5 |
| 13.283 | 863.9 |
| 13.846 | 408.8 |
| 14.063 | 59.0 |
| 15.895 | 383.8 |
| 16.209 | 493.0 |
| 17.330 | 1572.0 |
| 17.771 | 472.1 |
| 18.607 | 195.6 |
| 19.262 | 163.2 |
| 20.465 | 227.6 |
| 20.833 | 387.8 |
| 21.585 | 454.1 |
| 22.639 | 921.8 |
| 22.893 | 359.8 |
| 23.361 | 1365.2 |
| 23.781 | 121.2 |
| 24.245 | 977.9 |
| 24.477 | 177.1 |
| 25.071 | 104.2 |
| 25.734 | 255.7 |
| 25.983 | 207.3 |
| 26.202 | 444.2 |
| 26.597 | 183.4 |
| 27.734 | 140.1 |
| 28.339 | 114.4 |
| 28.904 | 107.3 |
| 29.685 | 100.6 |
| 30.515 | 236.1 |
| 31.499 | 195.2 |
| 32.090 | 300.9 |
| 32.935 | 105.7 |
| 33.296 | 142.7 |
| 37.042 | 148.3 |
| 37.671 | 104.6 |
| 38.433 | 108.3 |
| 40.335 | 91.2 |

### B. Infrared

The infrared absorption spectrum (KBr) is shown below in FIG. 2.

### Example 2

### Preparation of Rosiglitazone Base

130.3 g (366.6 mmol) of 5-{4-[2-(methylpyridin-2-ylamino)ethoxy]benzylidene}-thiazolidine-2,4-dione, 181.0 g (714.7 mmol) of diethyl 1,4-dihydro-2,6-dimethyl-3,5-pyridinedicarboxylate and 76.2 g of silica gel (0.063-0.200 mm particle size) were added over 885 mL of toluene. The resulting suspension was heated to reflux temperature for 24 hours, with azeotropical removal of water (from silica gel). The suspension was cooled down to 15° C. and was filtered using a Büchner funnel. The filter cake was washed with 300 mL of toluene. The solid was then suspended in 1.25 L of tetrahydrofuran. The mixture was heated to reflux temperature and cooled down to 30° C. Silica gel was removed by filtration (filter cake was washed with 125 mL of tetrahydrofuran). The filtered solution was concentrated by distillation of 875 mL of tetrahydrofuran under atmospheric pressure. The resulting solution was cooled down to 30° C. and non-reacted diethyl 1,4-dihydro-2,6-dimethyl-3,5-pyridinedicarboxylate was removed by filtration. 1 L of isopropanol was added to the filtered solution, and tetrahydrofuran was removed by distillation of 1 L of the solvent mixture, under atmospheric pressure. The resulting suspension was cooled down to 10° C. and filtered using a Büchner funnel. The filter cake was washed with 200 mL of isopropanol. 94.8 g (73% yield, 93.23% purity by HPLC) of Rosiglitazone base were obtained, m.p. 153.5-154.9° C.

The following characterization data was generated for the rosiglitazone base:

### A. X-Ray Powder Diffraction (XRPD)

The XRPD pattern is shown below in FIG. 3 and a summary of the characteristic XRPD angles (2θ°) and relative intensities (in %) are given in Table 2.

TABLE 2

| peak_position | peak_intensity |
|---|---|
| 12.580 | 68.0 |
| 13.568 | 602.8 |
| 13.996 | 1389.1 |
| 16.449 | 465.9 |
| 16.705 | 1396.4 |
| 17.004 | 372.4 |
| 17.354 | 1286.0 |
| 17.736 | 143.4 |
| 20.279 | 702.4 |
| 21.580 | 323.2 |
| 22.035 | 640.9 |
| 22.161 | 898.8 |
| 22.486 | 505.2 |
| 23.648 | 276.7 |
| 24.098 | 117.4 |
| 24.855 | 381.5 |
| 25.496 | 1220.9 |
| 26.180 | 497.5 |
| 26.395 | 407.1 |
| 27.588 | 212.8 |
| 28.225 | 141.2 |
| 29.028 | 145.4 |
| 29.602 | 104.8 |
| 29.832 | 131.3 |
| 30.116 | 189.7 |
| 31.525 | 336.2 |
| 34.232 | 131.3 |
| 35.427 | 143.2 |
| 35.587 | 116.7 |
| 38.828 | 114.9 |
| 40.489 | 109.5 |
| 45.801 | 78.3 |

### B. Infrared

The infrared absorption spectrum (KBr) is shown below in FIG. 4.

### Example 3

### Preparation of Rosiglitazone Maleate

93.3 g (261.0 mmol) of Rosiglitazone base as prepared in example 2 and 36.4 g (313.2 mmol) of maleic acid were suspended in 510 mL of isopropanol. The suspension was heated to reflux temperature for 1 hour. The initial suspension turned into a clear colorless solution. The resulting solution was cooled down to 10° C. and filtered using a Büchner funnel. The filter cake was washed with 200 mL of isopropanol. 105.0 g of crude Rosiglitazone maleate were obtained (85% yield, 99.57% purity by HPLC).

41.1 g (86.9 mmol) of the crude solid and 1.7 g (14.5 mmol) of maleic acid were suspended in 200 mL of ethanol (0.17 equivalents of maleic acid per 2.3 liters of ethanol which is approximately a maleic acid:ethanol ratio of about 13.5). The suspension was heated to reflux temperature for 20 minutes. The resulting solution was cooled down to 65° C. Seeding particles of the desired polymorph of Rosiglitazone maleate were added to the solution. The resulting suspension was cooled down to 0° C. and filtered using a Büchner funnel. The filter cake was washed with 50 mL of ethanol. 39.5 g of Rosiglitazone maleate were obtained (96% yield, 99.89% purity by HPLC), m.p. 120.6-121.7° C.

The rosiglitazone maleate was obtained typically having the following particle size distribution: D (v, 0.1): 1.1 to 1.5 μm; D (v, 0.5): 7.5 to 8.3 μm; D (v, 0.9): 37.5 to 51.0 μm; and typically having the mean value of the volume mean diameter of the particles within the range 14.0 to 18.0 μm.

The following characterization data was generated for the rosiglitazone maleate:

### A. Infrared

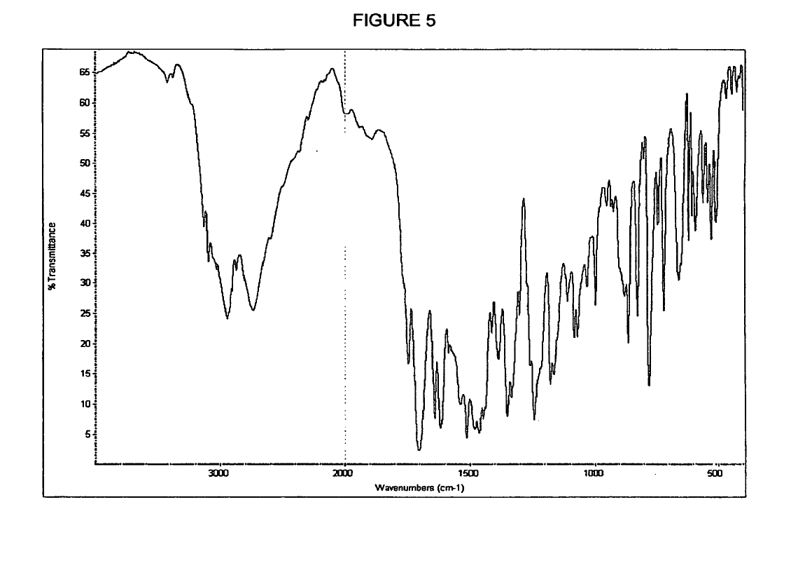

The infrared absorption spectrum (KBr) is shown below in FIG. 5 and a summary of the principal absorptions is given in Table 3.

TABLE 3
Peak positions (cm⁻¹)

| | | | |
|---|---|---|---|
| 3132 | 1514 | 1178 | 825 |
| 3101 | 1483 | 1163 | 778 |
| 2949 | 1464 | 1110 | 741 |
| 2741 | 1448 | 1082 | 717 |
| 1748 | 1414 | 1068 | 658 |
| 1705 | 1386 | 1031 | 617 |
| 1642 | 1352 | 998 | 604 |
| 1616 | 1335 | 952 | |
| 1586 | 1303 | 923 | |
| 1538 | 1245 | 862 | |

### B. X-Ray Powder Diffraction (XRPD)

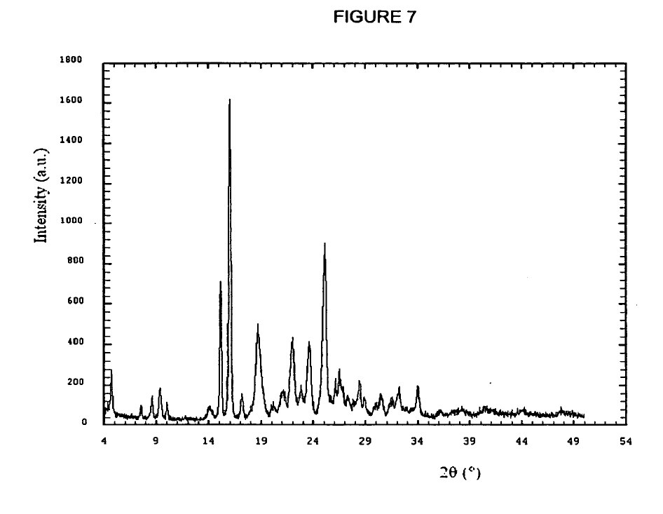

The XRPD pattern is shown in FIG. 7.

It will be apparent to those skilled in the art that various modifications and variations can be made in the present invention and specific examples provided herein without departing from the spirit or scope of the invention. Thus, it is intended that the present invention covers the modifications and variations of this invention that come within the scope of any claims and their equivalents.

1. A process for preparing the polymorphic form of 5-(4-[2-(N-methyl-N-(2-pyridyl)amino)ethoxy]benzylidene)-2,4-thiazolidinedione 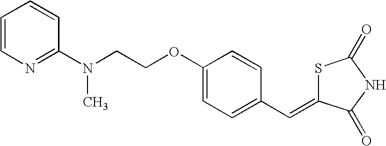
which comprises:
(a) mixing the compound 5-(4-[2-(N-methyl-N-(2-pyridyl)amino)ethoxy]benzylidene)-2,4-thiazolidinedione with a first organic solvent to form a solution;
(b) mixing a second organic solvent with the solution of step (a) to precipitate a polymorphic form of 5-(4-[2-(N-methyl-N-(2-pyridyl)amino)ethoxy]benzylidene)-2,4-thiazolidinedione
wherein the first organic solvent is an amido solvent and the second organic solvent is an alcohol.

2. The process of claim 1, wherein the first organic solvent is N,N-dimethylformamide (DMF); and the second organic solvent is isopropanol.

3. The process of claim 2, wherein the ratio of N,N-dimethylformamide (DMF)/isopropanol is about 1:2 to about 1:5.

4. The process of claim 3, wherein the polymorphic form of 5-(4-[2-(N-methyl-N-(2-pyridyl)amino)ethoxy]benzylidene)-2,4-thiazolidinedione has the X-ray powder diffraction (XRPD) characterized by the principal angles and relative intensities reported in FIG. 1.

5. The process of claim 3, wherein the polymorphic form of 5-(4-[2-(N-methyl-N-(2-pyridyl)amino)ethoxy]benzylidene)-2,4-thiazolidinedione has the infrared absorption spectrum characterized by the principal absorptions reported in FIG. 2.

6. A polymorphic form of the compound 5-(4-[2-(N-methyl-N-(2-pyridyl)-amino)ethoxy]benzylidene)-2,4-thiazolidinedione produced by the process of claim 1.

7. The polymorphic form of compound of claim 6 wherein the X-ray powder diffraction (XRPD) is characterized by the principal angles and relative intensities reported in FIG. 1.

8. The polymorphic form of compound of claim 6 wherein the infrared absorption spectrum is characterized by the principal absorptions reported in FIG. 2.

9-13. (canceled)

14. A polymorphic form of rosiglitazone produced by the process of claim 9.

15. The polymorphic form of rosiglitazone of claim 14, wherein the X-ray powder diffraction (XRPD) is characterized by the principal angles and relative intensities reported in FIG. 3.

16. The polymorphic form of rosiglitazone of claim 14, wherein the IR-spectrum is characterized by the principal absorptions reported in FIG. 4.

17. A process of preparing the polymorphic form of a rosiglitazone salt which comprises recrystallizing rosiglitazone salt with an organic acid of the salt in an alcohol solvent to form a polymorphic form of rosiglitazone salt, wherein the ratio of organic acid of the salt/rosiglitazone salt is about 1:2 to 1:20 by mole.

18. The process of claim 17, wherein the organic acid of the salt is a mono- or di-carboxylic acid, the alcohol solvent is a C₁-C₆ alcohol and the ratio of organic acid of the salt/rosiglitazone salt is about 1:5 to 1:15 by weight.

19. The process of claim 18, wherein the rosiglitazone salt is rosiglitazone maleate, the organic acid of the salt is maleic acid, the alcohol is ethanol and the ratio of organic acid of the salt/rosiglitazone salt is about 1:6 by mole.

20. The process of claim 17, wherein the particle size distribution of the polymorphic form of rosiglitazone salt is:
(1) about 5 to about 15% of the total volume of polymorphic form of rosiglitazone salt having a particle size of between about 1.0 micrometer to about 1.5 micrometers;
(2) about 45 to about 55% of the total volume of polymorphic form of rosiglitazone salt having a particle size of between about 7.5 micrometers to about 8.5 micrometers; and
(3) about 85 to about 95% of the total volume of polymorphic form of rosiglitazone salt having a particle size of between about 37.0 micrometers to about 55.0 micrometers.
